(19) United States
(12) Patent Application Publication
(10) Pub. No.: US 2026/0260713 A1
(43) Pub. Date: Sep. 3, 2026

(54) IDENTIFYING AN ANTI-AGGLOMERANT HYDRATE INHIBITOR

(71) Applicant: Schlumberger Technology Corporation, Sugar Land, TX (US)

(72) Inventors: Sijia Hu, Houston, TX (US); Simon Hunter, Houston, TX (US); Clare Schoene, Houston, TX (US); Chunfang Fan, Houston, TX (US); Ahmed Samy, Clamart (FR)

(21) Appl. No.: 19/067,100

(22) Filed: Feb. 28, 2025

(51) Int. Cl. G16C 20/30; G06F 30/28; G16C 20/70

(52) U.S. Cl. CPC G16C 20/30; G06F 30/28; G16C 20/70

(57) ABSTRACT

An inhibitor selection system may receive fluid properties of a fluid sample, the fluid sample including water and hydrocarbon gas. An inhibitor selection system may identify testing results having historical fluid properties with a higher severity than the fluid properties of the fluid sample, the testing results including tests of the historical fluid properties tested with a plurality of anti-agglomerant hydrate inhibitors. An inhibitor selection system may as a result of the historical fluid properties being more severe than the fluid properties of the fluid sample, pre-selecting at least one anti-agglomerant hydrate inhibitor. An inhibitor selection system may assign a rating to the at least one anti-agglomerant hydrate inhibitor. An inhibitor selection system may select, based on the rating, a selected anti-agglomerant hydrate inhibitor from the at least one anti-agglomerant hydrate inhibitor.

### BACKGROUND OF THE DISCLOSURE

Oil and gas production systems often transport a combination of water, hydrocarbons, and other compounds and gasses. In some situations, water and hydrocarbons may for hydrates. Hydrates may agglomerate in transportation pipelines and transportation facilities. This may reduce the efficiency and/or effectiveness of these facilities.

### SUMMARY

In some aspects, the techniques described herein relate to a method for selecting an anti-agglomerant hydrate inhibitor. An inhibitor model receives fluid properties of a fluid sample. The fluid sample includes water and hydrocarbons. The inhibitor model identifies, in a database, testing results having historical fluid properties with a higher severity than the fluid properties of the fluid sample. The testing results including tests of the historical fluid properties tested with a plurality of anti-agglomerant hydrate inhibitors. As a result of the historical fluid properties being more severe than the fluid properties of the fluid sample, the inhibitor model pre-selects at least one anti-agglomerant hydrate inhibitor. The inhibitor model assigns a rating to the at least one anti-agglomerant hydrate inhibitor. The inhibitor model selects, based on the rating, a selected anti-agglomerant hydrate inhibitor from the at least one anti-agglomerant hydrate inhibitor.

In some aspects, the techniques described herein relate to a method. An inhibitor model receives receiving water properties of a fluid sample of a production fluid and a hydrocarbon gas. The inhibitor model applies a machine learning model to the water properties. The machine learning model is trained to generate an output of at least one anti-agglomerant hydrate inhibitor based on an input of water properties. The machine learning model outputs a list of anti-agglomerant hydrate inhibitors for the fluid sample based on the water properties.

This summary is provided to introduce a selection of concepts that are further described in the detailed description. This summary is not intended to identify key or essential features of the claimed subject matter, nor is it intended to be used as an aid in limiting the scope of the claimed subject matter. Additional features and aspects of embodiments of the disclosure will be set forth herein, and in part will be obvious from the description, or may be learned by the practice of such embodiments.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to anti-agglomerant hydrate. Understanding that the drawings depict some example embodiments, the embodiments will be described and explained with additional specificity and detail through the use of the accompanying drawings in which:

FIG. 1 is a schematic representation of an anti-agglomerant hydrate inhibitor selection system, according to at least one embodiment of the present disclosure.

FIG. 2 is a flow diagram of an anti-agglomerant hydrate inhibitor selection system, according to at least one embodiment of the present disclosure.

FIG. 3 is a flow diagram of an anti-agglomerant hydrate inhibitor selection system, according to at least one embodiment of the present disclosure.

FIG. 4 shows a schematic view of a system to facilitate the selection of a suitable anti-agglomerant hydrate inhibitor for use with a specific production fluid, according to at least one embodiment of the present disclosure.

FIG. 5 is a flowchart of a method for selecting an anti-agglomerant hydrate inhibitor, according to at least one embodiment of the present disclosure.

FIG. 6 is a flowchart of a method for selecting an anti-agglomerant hydrate inhibitor, according to at least one embodiment of the present disclosure.

FIG. 7 is a representation of a computer system, according to at least one embodiment of the present disclosure.

### DETAILED DESCRIPTION

This disclosure generally relates to devices, systems, and methods for identifying and recommending anti-agglomerant hydrate inhibitors that may be compatible with a target fluid, the fluid including both water and hydrocarbons, such as a hydrocarbon gas. The hydrates of interest discussed herein include hydrates formed from methane, ethane, propane, carbon dioxide, nitrogen, oxygen, and so forth. The hydrates may form in water, and the hydrates may agglomerate at various locations in a pipeline or other transport facilities. Such hydrate formation and agglomeration may clog or otherwise obstruct pipelines, valves, or other elements of the pipeline or transport facility.

The agglomeration of hydrates may be reduced or prevented using an anti-agglomerant hydrate inhibitor. When hydrates form, they may be hydrophilic. This may cause droplets to merge, forming agglomerations that can clog pipelines and transportation facilities. An anti-agglomerant hydrate inhibitor may reduce the hydrophilic nature of the hydrates, thereby reducing or preventing the agglomeration of the hydrates. In some situations, the anti-agglomerant hydrate inhibitor may reduce the total size of hydrate agglomerate. In some situations, the anti-agglomerant hydrate inhibitor may cause the hydrate droplets to become hydrophobic, thereby preventing attachment of hydrate particles to each other.

Anti-agglomerant hydrate inhibitors are typically identified by testing multiple products, and then selecting the anti-agglomerant hydrate inhibitor with the best performance. However, such testing is costly and time-consuming. The anti-agglomerant hydrate inhibitors tested may be selected based on information in a product catalog, combined with a technician’s knowledge and expertise. This may result in the operator testing an excessive number of products. The results of various anti-agglomerant hydrate inhibitor tests may be stored in a test database, resulting in a large amount of historical data. But due to the number of products to be tested, it may be difficult to identify the most effective anti-agglomerant hydrate inhibitor.

In accordance with at least one embodiment of the present disclosure, an anti-agglomerant hydrate inhibitor selection system may extrapolate the historical data into a form that allows reliable predictions to be made about the interactions between production fluids and different anti-agglomerant hydrate inhibitors. The anti-agglomerant hydrate inhibitor selection system may identify correlations between the properties of water in the fluid, the gasses present in the production system, and the properties of anti-agglomerant hydrate inhibitors.

In some embodiments, to select an anti-agglomerant hydrate, a screening model may identify, in a database, testing results with historical fluid properties that are more severe than the fluid properties of the target production fluid. For example, hydrates may more readily form and agglomerate when the fluid properties of a production fluid are more severe, such as when the API gravity is greater, when the water cut is higher, or when the salinity is higher. The screening model may identify or pre-select, in the testing results, all of the anti-agglomerant hydrate inhibitors that have one or more properties that are more severe than the target production fluid.

In some embodiments, a rating model may rate the anti-agglomerant hydrate inhibitors that were pre-selected by the screening model. For example, the rating model may provide an effectiveness rating for the anti-agglomerant hydrate inhibitors based on properties of a fluid that are known to be influenced by the anti-agglomerant hydrate inhibitor and/or properties of an anti-agglomerant hydrate inhibitor that are known to influence the agglomeration of one or more hydrates. In some embodiments, the rating model may identify a number of rating factors that impact the overall effectiveness rating of the anti-agglomerant hydrate inhibitors. The rating model may identify a weight for each of the rating factors and generate the overall effectiveness rating based on the weighted combination of all of all of the rating factors.

In some embodiments, the anti-agglomerant hydrate inhibitor selection system may output a list of anti-agglomerant hydrate inhibitors. The list of anti-agglomerant hydrate inhibitors may be output based on which of the anti-agglomerant hydrate inhibitors have the highest effectiveness rating. In some embodiments, the anti-agglomerant hydrate inhibitor selection system may may provide all of the anti-agglomerant hydrate inhibitors that have an effectiveness rating that is higher than a threshold effectiveness rating. In some embodiments, the anti-agglomerant hydrate inhibitor selection system may the top several (e.g., 2, 3, 4, 5, 6, 7, 8, 9, 10) anti-agglomerant hydrate inhibitors based on the effectiveness rating. In some embodiments, the anti-agglomerant hydrate inhibitor selection system may output the single highest rated anti-agglomerant hydrate inhibitor. An operator may then select, from the outputted list of anti-agglomerant hydrate inhibitors, one or more anti-agglomerant hydrate inhibitors to use in an oil and gas production system and/or to test prior to use.

In some embodiments, a method for selecting anti-agglomerant hydrate inhibitors that have a high effectiveness rating for a target production fluid may include a user inputting one of more known properties of a target production fluid into a machine learning model that has been trained by a historical data set that includes one or more properties of a plurality of production fluids, one or more properties of a plurality of anti-agglomerant hydrate inhibitors, an associated hydrate type or hydrate category, and the effectiveness of the anti-agglomerant hydrate inhibitor with the associated production fluids. Historical data also may include testing conditions of anti-agglomerant hydrate inhibitors with production fluids thermodynamic properties (e.g., pressure and temperature), the testing apparatus used, and set-up and take down procedures of each test. The historical data set includes test results of a combination of a production fluid and an anti-agglomerant hydrate inhibitor. The machine learning model extrapolates unknown properties (e.g., unknown correlations between parameters) of the target production fluid and data sets within the historical data set, such as connections or relationships between the production fluid and properties of the anti-agglomerant hydrate inhibitor. The machine learning model predicts an effectiveness rating of the anti-agglomerant hydrate inhibitors based on the historical data set and extrapolated unknown properties for the one or more anti-agglomerant hydrate inhibitors that may be used with the target production fluid. The machine learning model outputs one or more of a list of production fluids having one or more properties within a numerical tolerance of the properties of the target production fluid, a list of anti-agglomerant hydrate inhibitors for use with the target production fluid; a list of anti-agglomerant hydrate inhibitor properties.

A user or the machine learning model may select or design a chosen anti-agglomerant hydrate inhibitor, based on the output, for use with the target production fluid. In one embodiment, the user may test a plurality of the output anti-agglomerant hydrate inhibitors with the target production fluid. Based on the actual test data, the user selects the anti-agglomerant hydrate inhibitor to be used with the target production fluid. Once selected, a user may validate the predicted anti-agglomerant hydrate inhibitor effectiveness rating with testing on the anti-agglomerant hydrate inhibitor and the target production fluid, and add the results of the actual testing to the historical data set.

The one or more properties of a plurality of production fluids may include a location of a source of one of the plurality of production fluids. The location of a source of a production fluid may allow the machine learning model to identify additional data sets within the historical data that may be closely related to the production fluid and facilitate the identification of effective anti-agglomerant hydrate inhibitors. The one or more properties of a plurality of production fluids may also include dissolved ions in the production fluids, such as sodium (Na), potassium (K), magnesium (Mg), calcium (Ca), Strontium (Sr), barium (Ba), iron (Fe), zinc (Zn), lead (Pb), chloride (Cl⁻), sulphate (SO₄²⁻), fluoride (F⁻), bromide (Br⁻), silica (SiO₂), bicarbonate (HCO₃⁻). In some embodiments, the one or more properties of the production fluids may include the hydrocarbon content of the fluid, including methane, ethane, propane, hydrocarbons of any carbon chain length, gasses including carbon dioxide, carbon monoxide, nitrogen, oxygen, hydrogen sulfide, and so forth. In some embodiments, the one or more properties of the production fluids may include bulk properties of the fluid, such as the total water cut or oil cut, the API gravity, the degree of subcooling, the salinity of the water, the total dissolved solids, the hydrocarbon liquid type, the hydrocarbon gas type, the hydrate type, the gas-to-liquid ratio, the liquid volume loading in the pipe, the operating temperature, the operating pressure, any other property, and combinations thereof, including inclusions and exclusions of any of the foregoing.

In some embodiments, the properties of the anti-agglomerant hydrate inhibitors may include one or more of overall minimum effective dosage, minimum effective dosage for a particular hydrate type, environmental rating, biodegradability, bioaccumulation, toxicity, Deepwater qualification status, pressure temperature and gas composition, product formulation, various test results, product cost, product location, product shipping time or complexity, product availability, lead time, supply chain considerations, any other inhibitor properties, and combinations thereof.

In some embodiments, a user or a machine learning model may add, to a historical database, records of production fluids and anti-agglomerant hydrate inhibitors. Each record includes one or more of properties of the production fluid including identification of a source of a production fluid, anti-agglomerant hydrate content, anti-agglomerant hydrate inhibitor identification, anti-agglomerant hydrate type, effectiveness rating, field operating conditions, and so forth. The machine learning model generates first model parameters to compare a target production fluid to the production fluids in the database. The machine learning model generates second model parameters to compute an anti-agglomerant hydrate inhibitor efficiency of the anti-agglomerant hydrate inhibitors in the database with the target production fluid and generates third model parameters to compute an effectiveness rating of the anti-agglomerant hydrate inhibitor in the database with the target production fluid. The machine learning model inputs properties of the target production fluid into a model containing the first model parameters, the second model parameters, and the third model parameters. The machine learning model then outputs one or more of a list of production fluids having one or more properties within a numerical tolerance of the properties of the target production fluid, a list of anti-agglomerant hydrate inhibitors for the target production fluid, each including an anti-agglomerant hydrate inhibitor effectiveness rating, and a list of inhibitor properties.

The machine learning model or a user may select or design a chosen anti-agglomerant hydrate inhibitor, based on the output, for use with the target production fluid. In one embodiment, the user may test a plurality of the output anti-agglomerant hydrate inhibitors with the target production fluid. Based on the actual test data, the user selects the anti-agglomerant hydrate inhibitor to be used with the target production fluid. Once selected, a user may validate the confidence level with testing on the anti-agglomerant hydrate inhibitor and the target production fluid and add the results of the actual testing to the historical data set. The historical data set may include test results of a combination of a production fluid and an anti-agglomerant hydrate inhibitor.

As illustrated by the foregoing discussion, the present disclosure utilizes a variety of terms to describe features and advantages of the anti-agglomerant hydrate inhibitor selection system. Additional detail is now provided regarding the meaning of such terms. For example, as used herein, a “production fluid” may include a substance that is collected from an oil and gas system. For instance, a “production fluid” may include a single phase of matter, such as a gas or a liquid. In some embodiments, a production fluid may include a combination of multiple materials and/or multiple phases of matter, such as a liquid having suspended solids, a liquid having entrained gas or solids, a liquid having gas bubbles, a gas suspended liquid droplets or solid particles. The production fluid may include a combination two or three of gas (e.g., natural gas), water (e.g., a brine including dissolved compounds), and oil (e.g., crude oil), including a mixture of gas and water, a mixture of oil and water, or a mixture of gas, oil, and water. The production fluid may include one or more compounds, including compounds dissolved in water, a mixture of different hydrocarbons, compounds dissolved in crude oil, or a mixture of two or more gasses.

As used herein, the term “hydrate” refers to a compound or a complex consisting of a three-dimensional lattice of hydrogen bonded water, with common gas molecules such as methane, ethane, carbon dioxide, hydrogen sulfide, and so forth, formed at high-pressure and low-temperature conditions. The formation of a hydrate in subsea oil and gas may be impacted by the natural gas composition of a production line, the composition and the presence of water, the presence of other gas or liquid hydrocarbons, the presence of non-hydrocarbon gasses, the dew point of the production line, the temperature, the pressure, any other factors, and combinations thereof. Hydrates may agglomerate to form solid, flammable crystals. Hydrates may agglomerate at various locations of a pipeline or processing facility, including the inner surface of a pipeline, valves, junctures, and so forth.

As used herein, the term “anti-agglomerant hydrate inhibitor” may refer to a chemical, compound, or additive that may be added to a production fluid to reduce the likelihood of hydrate agglomeration, or prevent altogether hydrate agglomeration. An anti-agglomerant hydrate inhibitor may reduce the likelihood of hydrate agglomeration by adjusting how hydrophilic or hydrophobic the resulting hydrates are.

As used herein, the term “machine learning” refers to algorithms that generate data-driven predictions or decisions from known input data by modeling high-level abstractions. Examples of machine-learning models include computer representations that are tunable (e.g., trainable) based on inputs to approximate unknown functions. For instance, a machine-learning model includes a model that utilizes algorithms to learn from, and make predictions on, known data by analyzing the known data to learn to generate outputs that reflect patterns and attributes of the known data. For example, machine-learning models include latent Dirichlet allocation (LDA), multi-arm bandit models, linear regression models, classification models, logistical regression models, random forest models, support vector machines (SVMs) models, neural networks (convolutional neural networks, recurrent neural networks such as LSTMs, graph neural networks, etc.), or decision tree models.

A machine learning model may be tuned (e.g., trained) based on training input to approximate unknown functions. For example, a machine learning model may refer to a neural network or other machine learning algorithm or architecture that learns and approximates complex functions and generate outputs based on a plurality of inputs provided to the machine learning model. In some embodiments, a machine learning model may include one or multiple machine learning models that cooperatively generate one or more outputs based on corresponding inputs.

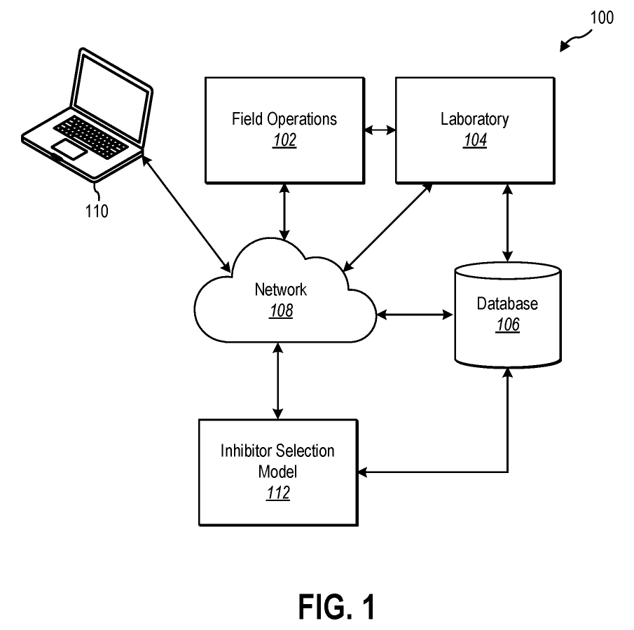

FIG. 1 is a schematic representation of an anti-agglomerant hydrate inhibitor selection system 100, according to at least one embodiment of the present disclosure. The anti-agglomerant hydrate inhibitor selection system 100 may facilitate the identification and selection of an anti-agglomerant hydrate inhibitor. Fluid samples may be collected from one or more field operations 102. One or more of the fluid samples may include a production fluid. The field operations 102 may include any field operation in an oil and gas exploration and production system. For example, the field operations 102 may include wellbore drilling, wellbore completion, wellbore production, wellbore pipelines and associated equipment, any other field operations, and combinations thereof. An operator may collect fluid samples from the field operations 102. For example, the operator may collect a fluid sample of fluids pumped into and subsequently retrieved from a wellbore (e.g., drilling fluid or drilling mud, hydraulic fracturing fluid, artificial lift fluid). In some examples, the operator may collect a fluid sample of fluids produced by a wellbore, such as water and/or oil.

A laboratory 104 may receive the fluid sample from the field operations 102. The laboratory 104 may perform one or more tests on the fluid. For example, the laboratory 104 may perform tests to determine the composition of the fluid, the physical properties of the fluid, the chemical properties of the fluid, and so forth. In some embodiments, the laboratory 104 may determine the likelihood that a hydrate will form and agglomerate based on the composition and mixture of the production fluid. In some examples, the laboratory 104 may analyze the impact of one or more additives on the fluid. For example, the laboratory 104 may analyze the impact of adding an anti-agglomerant hydrate inhibitor on the formation and agglomeration of hydrates in the production system.

In some embodiments, a single laboratory 104 may analyze the fluid sample collected from the field operations 102. In some embodiments, the field operations 102 may send the fluid sample to multiple laboratories 104, with different laboratories 104 performing different tests on the fluid sample.

The laboratory 104 may generate one or more lab reports associated with the tests performed on the fluid sample. The lab reports may include data stored in one or more data fields. For example, the lab reports may include the testing conditions, the properties of the fluid sample, the anti-agglomerant hydrate inhibitor tested, the testing results, including hydrate types and hydrate agglomeration tendencies, and information associated with the source of the sample. In some embodiments, the field operations 102 may include field operating conditions, or operational information associated with collecting the fluid sample, such as temperatures, pressures, wellbore identification, wellbore location, relevant geology, collection equipment, collection crew, wellbore owner information, and so forth.

As discussed herein, the lab reports may be generated to information operational decisions. For example, the lab reports may be generated to inform the selection of an additive to a fluid stream. Typically, when an operator desires to make an operational decision, the operator may request the collection and analysis of the fluid samples. However, such analysis may be expensive and time consuming. The operator may desire to make the operational decision based tests previously performed on other fluid samples, including fluid samples from similar wellbores and/or dissimilar wellbores. Such historical data may be stored in a database 106. The database 106 may include lab reports from various laboratories 104.

The field operations 102, the laboratory 104, and the database 106 may be connected over a network 108, such as the internet. For example, the field operations 102 may collect the sample, record information associated with the collection of the sample in a field report, and upload the field report to the database 106 over the network 108. The field operations 102 may physically deliver the fluid sample to the laboratory 104, and the laboratory 104 may generate the lab report and upload the lab report to the database 106 over the network 108. In some embodiments, the database 106 may be located on a remote server, such as a cloud server, and the field operations 102 and/or the laboratory 104 may upload the lab reports in real time to the database 106. In some embodiments, the field operations 102 and/or the laboratory 104 may upload the lab reports to the database 106 after completion of multiple lab reports, or when internet access is available to the operator. A user may access the lab reports from the database 106 using a user device 110 via the network 108. In some embodiments, the user may further be in communication with the field operations 102 and/or the laboratory 104 over the network 108.

In accordance with at least one embodiment of the present disclosure, an inhibitor model 112 may prepare an output of a recommended anti-agglomerant hydrate inhibitor based on an inputted target production fluid. The inhibitor model 112 may receive sample fluid properties of a sample of a production fluid from the laboratory 104. The inhibitor model 112 may pre-select, from the database 106, a list of one or more selected anti-agglomerant hydrate inhibitors. The inhibitor model 112 may pre-select the list of the anti-agglomerant hydrate inhibitors based on the sample fluid properties. For example, the inhibitor model 112 may pre-select anti-agglomerant hydrate inhibitors based on whether the tested fluid properties in the database 106 are more severe than the sample fluid properties of the sample production fluid. Fluid properties that are more “severe” may be fluid properties that encourage or facilitate faster formation of hydrate agglomerates or formation of larger hydrate agglomerates. Pre-selecting based on testing conditions of anti-agglomerant hydrate inhibitors that successfully inhibit the formation of hydrate agglomerates may help to ensure that a particular anti-agglomerant hydrate inhibitor will be effective. For example, an anti-agglomerant hydrate inhibitor that is effective in more severe conditions will likely be effective in less severe conditions. Pre-selecting anti-agglomerant hydrate inhibitors based on condition severity may help to reduce the testing or other analysis of the anti-agglomerant hydrate inhibitors.

In some embodiments, the inhibitor model 112 may assign an effectiveness rating to list the anti-agglomerant hydrate inhibitors for the identified fluid parameters. The effectiveness rating may be an indication of how well the anti-agglomerant hydrate inhibitor may inhibit the agglomeration of hydrates in the production system. The rating may be based on one or more rating factors. The rating factors may be associated with the testing results, including the dosage of the anti-agglomerant hydrate inhibitor, the number of tests performed with the anti-agglomerant hydrate inhibitors, the difference between the test fluid parameters and the sample fluid parameters, subcooling temperature compared to the field condition, regulatory registrations, such as REACH, DSL, TSCA, and so forth, biodegradability, bioaccumulation, toxicity, deepwater qualification, cost, product availability, compatibility, any other rating factors, and combinations thereof. As a specific, non-limiting example, compatibility is important because testing an anti-agglomerant may utilize other chemicals to isolate certain hydrates or agglomeration conditions. For example, wax (e.g., paraffin) and hydrates may both form at low temperature, so an operator may add a paraffin inhibitor to the fluid, and the anti-agglomerant hydrate inhibitor and the paraffin inhibitor may be compatible to prevent undesirable results. Similarly, scale may form due to the brine, and a scale inhibitor may be compatible with the anti-agglomerant hydrate inhibitor. Thus, the compatibility rating of the anti-agglomerant hydrate inhibitor may be used as a rating factor for selection purposes.

The inhibitor model 112 may generate an overall effectiveness rating for each of the anti-agglomerant hydrate inhibitors based on the sample fluid parameters. The overall effectiveness rating may be a combination of the rating factors. In some embodiments, the inhibitor model 112 may apply a weight to the rating factors. For example, the inhibitor model 112 may apply a weight to the rating factors based on their impact on the overall effectiveness rating. In some examples, all of the rating factors may have the same weight. In some embodiments, different rating factors may have different weights.

Other inputs to the inhibitor model 112 may include an anti-agglomerant hydrate catalog. The anti-agglomerant hydrate product catalog may be a representation of the available anti-agglomerant hydrate inhibitors. For example, the anti-agglomerant hydrate product catalog may include a listing of anti-agglomerant hydrate inhibitors and properties thereof. The properties of the anti-agglomerant hydrate inhibitor may include chemistry type, composition, raw material code, type of treated anti-agglomerant hydrate, regional availability, environmental data, viscosity, thermal stability, and so forth. In some embodiments, the properties of the anti-agglomerant hydrate inhibitor may be provided by the manufacturer. In some embodiments, the properties of the anti-agglomerant hydrate inhibitor may be altered, amended, or otherwise added to by the laboratory 104. In some embodiments, the anti-agglomerant hydrate inhibitor selection system 100 may develop one or more derived features based on the interaction between the anti-agglomerant hydrate inhibitor properties and the properties of the water samples stored in the database 106. In some embodiments, the historical data in the database 106 may include test results from a particular anti-agglomerant hydrate inhibitor, the properties of the anti-agglomerant hydrate inhibitor, and the impact of the anti-agglomerant hydrate inhibitor on the water sample.

In accordance with at least one embodiment of the present disclosure, the inhibitor model 112 may include a machine learning model. The machine learning model may be trained to select at least one anti-agglomerant hydrate inhibitor that may be applicable to the sample production fluid. For example, the inhibitor model 112 may be trained based on historical data from the database 106. The inhibitor model 112 may identify or extrapolate unknown properties of the historical data in the database 106, including correlations between anti-agglomerant hydrate inhibitors, water properties, and field operating conditions. The inhibitor model 112 may be trained to output a list of one or more anti-agglomerant hydrate inhibitors. The outputted anti-agglomerant hydrate inhibitors may then be applied at the field operations 102 to reduce anti-agglomerant hydrate formation and deposition in the oil and gas systems.

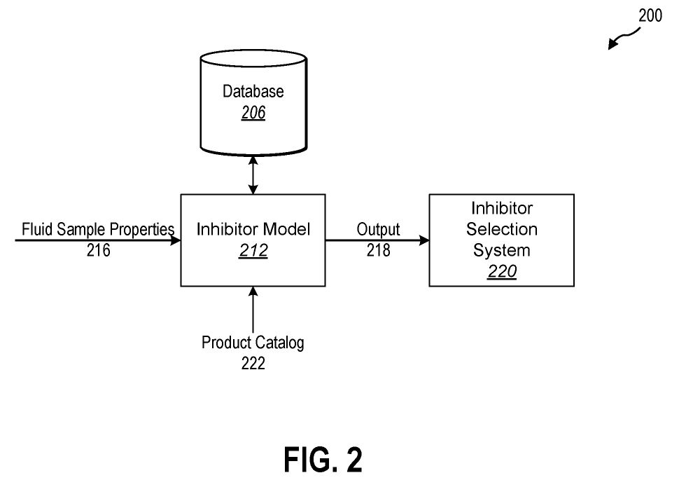

FIG. 2 is a flow diagram of an anti-agglomerant hydrate inhibitor selection system 200, according to at least one embodiment of the present disclosure. In the anti-agglomerant hydrate inhibitor selection system 200, an inhibitor model 212 may receive fluid sample properties 216. The inhibitor model 212 may receive fluid sample properties of a sample collected from a production fluid. The inhibitor model 212 may analyze the fluid sample properties 216 to generate an output 218 that includes one or more anti-agglomerant hydrate inhibitors. An inhibitor selection system 220 may analyze the output of the one or more anti-agglomerant hydrate inhibitors to select a chosen anti-agglomerant hydrate inhibitor for use. In some embodiments, the inhibitor selection system 220 may select a list of selected anti-agglomerant hydrate inhibitors for a user to choose from.

The inhibitor model 212 may generate the output 218 by analyzing historical data from the database 206. For example, the inhibitor model 212 may generate the output 218 by identifying historical test results in the database 206 that tested anti-agglomerant hydrate inhibitors under fluid conditions that were more severe than the fluid sample properties 216. In some examples, the inhibitor model 212 may generate the output 218 by identifying an effectiveness of the tested anti-agglomerant hydrate inhibitors on similar conditions to the fluid sample properties 216.

The inhibitor selection system 220 may receive the output 218 and arrange or order the anti-agglomerant hydrate inhibitors for selection by the user. For example, the output 218 may include an effectiveness rating. The inhibitor selection system 220 may organize, sort, or otherwise arrange the outputted anti-agglomerant hydrate inhibitors to present to a user. In some embodiments, the inhibitor selection system 220 may select the best anti-agglomerant hydrate inhibitors to provide to the user.

In some embodiments, the inhibitor model 212 and/or the inhibitor selection system 220 may include a ranking model. As discussed herein, the anti-agglomerant hydrate inhibitors identified from the database 206 may be ranked with an effectiveness ranking. The effectiveness ranking may be a representation of the performance of the anti-agglomerant hydrate inhibitors. The effectiveness ranking may be based on one or more ranking factors, as discussed herein.

In some embodiments, the inhibitor model 212 may include a machine learning model. The machine learning model may be trained on historical data from a database 206. As discussed herein, the database 206 may include historical lab testing results, including fluid sample properties, test type, tested anti-agglomerant hydrate inhibitor, anti-agglomerant hydrate inhibitor properties, test results, and so forth. During training, the machine learning model may extrapolate unknown properties of the historical data, which may include connections, relationships, ratios, and so forth between the historical data.

In some embodiments, the inhibitor model 212 may receive, as part of the input, a product catalog 222 of various anti-agglomerant hydrate inhibitors. The product catalog 222 may include the identification of anti-agglomerant hydrate inhibitors, as well as known properties of the anti-agglomerant hydrate inhibitors. In some embodiments, the product catalog 222 may include anti-agglomerant hydrate inhibitors that have been used in the recorded historical data of the database 206. In some embodiments, the product catalog 222 may include anti-agglomerant hydrate inhibitors that not been used in the recorded historical data of the database 206.

In some examples, the product catalog 222 may be an indication or instruction to the inhibitor model 212 of the anti-agglomerant hydrate inhibitors that are available. For example, the product catalog 222 may provide an indication of the anti-agglomerant hydrate inhibitors that are available within a certain geographical area, available based on supplier relations, available based on stock constraints, or otherwise available.

The output 218 generated by the inhibitor model 212 may include any type of anti-agglomerant hydrate inhibitor output. For example, the output 218 may include a recommendation of an anti-agglomerant hydrate inhibitor that has previously been used in the historical data stored in the database 206. In some embodiments, the output 218 may include a recommendation of an anti-agglomerant hydrate inhibitor that has been used in the database 206 and is also provided to the inhibitor model 212 as available based on the product catalog 222.

In some embodiments, the inhibitor model 212 may provide the output 218 as a set of recommended anti-agglomerant hydrate inhibitor parameters. The inhibitor selection system 220 may review the recommended anti-agglomerant hydrate inhibitor parameters and select an anti-agglomerant hydrate inhibitor from the product catalog 222 based on the recommended anti-agglomerant hydrate inhibitor properties. In some embodiments, none of the anti-agglomerant hydrate inhibitors from the product catalog 222 are within a particular effectiveness rating range or are compatible with the production fluid. For unidentified anti-agglomerant hydrate inhibitors, the inhibitor model 212 may output the list of anti-agglomerant hydrate inhibitor properties. A user may request that a manufacturer design or recommend an anti-agglomerant hydrate inhibitor that is compliant with the identified properties.

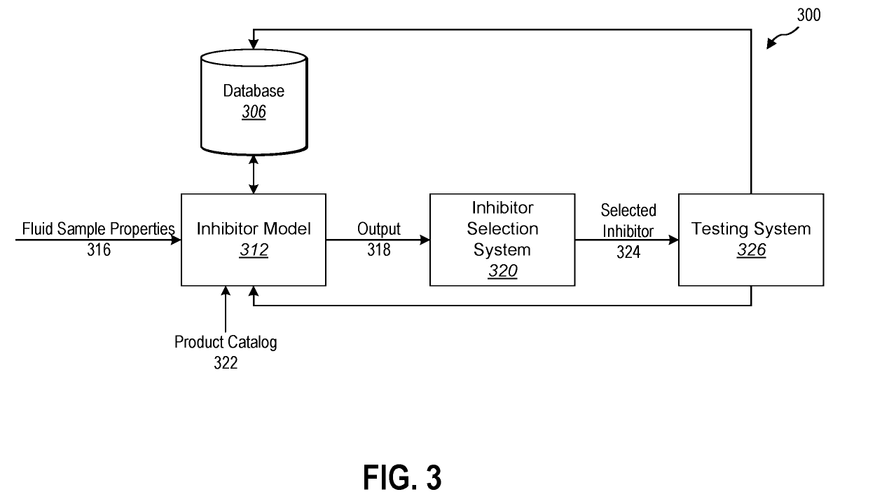

FIG. 3 is a flow diagram of an anti-agglomerant hydrate inhibitor selection system 300, according to at least one embodiment of the present disclosure. In the anti-agglomerant hydrate inhibitor selection system 300, an inhibitor model 312 may receive fluid sample properties 316. The inhibitor model 212 may receive fluid sample properties of a sample collected from a production fluid, analyze the fluid sample properties 216, and generate an output 218 that includes one or more anti-agglomerant hydrate inhibitors. An inhibitor selection system 220 may analyze the output of the one or more anti-agglomerant hydrate inhibitors to select a list of anti-agglomerant hydrate inhibitors for testing and/or use. The inhibitor model 212 may generate the output 218 by analyzing historical data from the database 206.

The inhibitor selection system 220 may receive the output 218 and arrange or order the anti-agglomerant hydrate inhibitors for selection by the user. For example, the output 218 may include an effectiveness rating. The inhibitor selection system 220 may organize, sort, or otherwise arrange the outputted anti-agglomerant hydrate inhibitors to present to a user. In some embodiments, the inhibitor selection system 220 may select the best anti-agglomerant hydrate inhibitors to provide to the user.

In some embodiments, the inhibitor model 212 and/or the inhibitor selection system 220 may include a ranking model. As discussed herein, the anti-agglomerant hydrate inhibitors identified from the database 206 may be ranked with an effectiveness ranking. The effectiveness ranking may be a representation of the performance of the anti-agglomerant hydrate inhibitors. The effectiveness ranking may be based on one or more ranking factors, as discussed herein.

As discussed herein, the inhibitor model 312 may include a machine learning model trained on historical data from a database 306, which may include historical lab testing results, including fluid sample properties, historical fluid properties of historical samples, test type, tested anti-agglomerant hydrate inhibitor, anti-agglomerant hydrate inhibitor properties, test results, and so forth.

In some embodiments, one or more selected anti-agglomerant hydrate inhibitors 324 may be tested at a testing system 326. For example, the testing system 326 may test the recommended or selected anti-agglomerant hydrate inhibitors 324 and determine their applicability to the production fluid that originated the fluid sample properties 316. As discussed herein, the inhibitor model 312 may pre-select certain anti-agglomerant hydrate inhibitors. This may reduce the total amount of testing the testing system 326 may need to perform, thereby reducing the time and/or money expended to identify and select an anti-agglomerant hydrate inhibitor.

In accordance with at least one embodiment of the present disclosure, the test results from the testing system 326 may be provided to the inhibitor model 312 and/or the database 306. The inhibitor model 312 may be re-trained and/or fine-tuned based on the test results from the selected anti-agglomerant hydrate inhibitors 324 and the fluid sample properties 316. This may help to further improve the accuracy and/or relevance of the results of the inhibitor model 312.

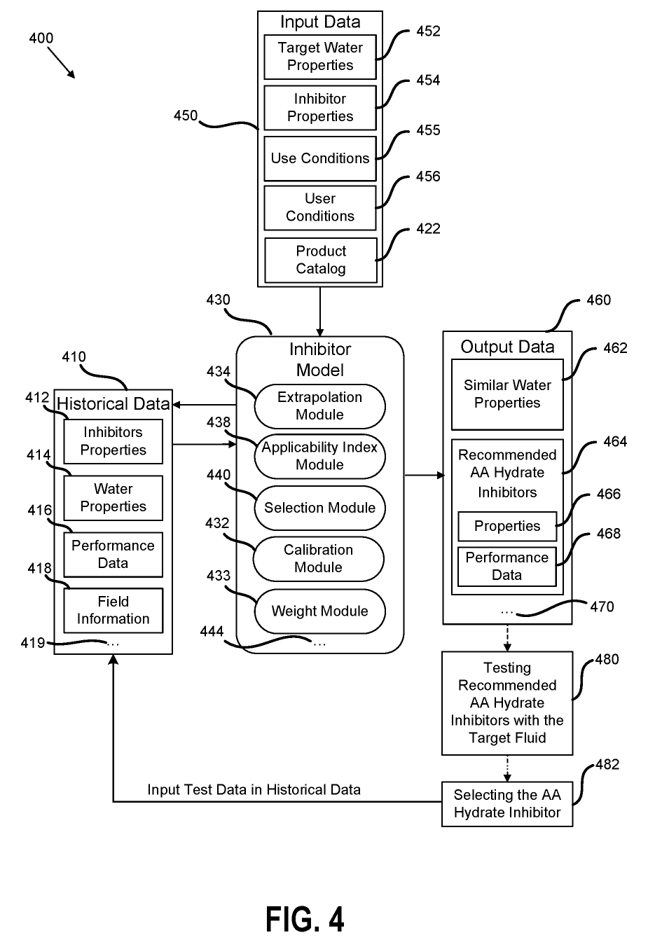

FIG. 4 shows a schematic view of a system 400 to facilitate the selection of a suitable anti-agglomerant hydrate inhibitor for use with a specific production fluid, according to at least one embodiment of the present disclosure. As shown, anti-agglomerant hydrate inhibitors properties 412 and the production fluids properties 414 may be part of a historical database or more generally form part of historical data 410. Additionally, the historical data 410 may include performance data 416, which includes the parameters, procedures, and results of anti-agglomerant hydrate inhibitor tests and other water content tests. Performance data 416 may also include the mechanical or crystalline properties of anti-agglomerant hydrate formations, including anti-agglomerant hydrate formations of production fluids having anti-agglomerant hydrate inhibitors mixed in at different dosages. Performance data 416 may include a wide variety of experiment procedures and the resulting test data. Unsuccessful test data may also be included in the historical data 410 to provide diverse performance profiles and may allow the inhibitor model 430 to identify potential areas for tests and data to be gathered.

The historical data 410 may also include field information 418, such as in field reports of anti-agglomerant hydrate build up in equipment, journal articles, published information, and the current and historical pricing of anti-agglomerant hydrate inhibitors, precursor materials, and manufacturing costs. Anticipated field conditions for use of an anti-agglomerant hydrate inhibitor with a target production fluid may be included to inform selection of test conditions for inclusion in the inhibitor model 430. Field information 418 may include environmental and field conditions such as production fluid bulk temperature, pipeline outer wall temperature, weather conditions around a pipeline or production location, flow rates, and pipeline and equipment materials and associated surface friction. Other information 419 not related to the use of anti-agglomerant hydrate inhibitors may also be included in the historical data, such as weather reports for specific geographic locations, maps, and geologic formation data.

The system 400 includes an inhibitor model 430. The inhibitor model 430 uses the historical data 410 to identify potential correlations and similarities in the historical data to make recommendations of potential anti-agglomerant hydrate inhibitors that may be used with a target production fluid. In some embodiments, the inhibitor model 430 may be constructed as a model to which machine learning techniques can be applied. In one embodiment, if the inhibitor model 430 is to be used to select a list of inhibitors that may be used with a target production fluid, the inhibitor model 430 may be a classification type model. If the model is to be used to predict an effectiveness rating for a pairing of a target production fluid with a selected anti-agglomerant hydrate inhibitor, the inhibitor model 430 may be a regression type model. The inhibitor model 430 may include modules that perform specific types of evaluations. For example, one module may perform a classification type evaluation while another module performs a regression type evaluation. For example, one module may select a list of inhibitors likely to be successful for a target production fluid, and another module may predict inhibition efficiency for each of the selected inhibitors with the target production fluid. For example, the model can contain any or all of a KNN algorithm, a random forest algorithm, a Bayesian network algorithm, a response surface algorithm, a fractional factorial algorithm, or other similar algorithms.

As shown, the inhibitor model 430 may include multiple modules, including an extrapolation module 434, a ranking module 438, a selection module 440, a calibration module 432, and other modules 444. The inhibitor model 430 may be a machine learning model, a large language model, or use artificial intelligence to train itself on the historical data 410 to identify potential correlations between the anti-agglomerant hydrate inhibitors properties 412 and the production fluids properties 414 using the performance data 416, and in some embodiments, the field information 418.

The extrapolation module 434 of the inhibitor model 430 may extrapolate the historical data 410 to populate unknown properties and information in the historical data 410. The extrapolation module 434 may use the historical data 410 to populate unknown properties and information in the historical data 410 by borrowing and extrapolating known data from data sets. The extrapolation module 434 may also use new data input into the historical data 410 to further refine its extrapolations and may periodically update the extrapolated data based on new input data. This process over time may further refine the model and allow better correlations to be developed in the extrapolation module 434.

In some embodiments, the extrapolation module 434 may use the input data 450 and the historical data 410 to extrapolate the information in input data 450 to fill in any information missing from the input data 450. By extrapolating the input data, the model may be better able to identify information in the historical data 410 that may be relevant to the input data 450.

The ranking module 438 may be used to rank recommended anti-agglomerant hydrate inhibitors to the associated production fluid. For example, the ranking module 438 may prepare a numerical representation of the expected performance or applicability of the anti-agglomerant hydrate inhibitor based on the performance data 416 from the historical data 410.

The selection module 440 may select the output data 460 selected by the inhibitor model 430 that meets a predetermined set of criteria, such as a particular effectiveness ranking. The selection module 440 may organize the output data 460. For example, the selection model 440 may organize the output data 460 with the highest anti-agglomerant hydrate effectiveness ranking first. In some examples, the selection model 440 may organize the output data 460 in any other manner, such as by price, availability, location of a stockpile of the anti-agglomerant hydrate inhibitor, amount of anti-agglomerant hydrate inhibitor on site, dosage rate, and so forth.

Other modules 444 may also be used by the inhibitor model 430. For example, a mapping module may use the source location data and geological information of a production fluid to collate data sets that may share or be near the source location and may be from the same geological formation. The mapping module may identify the geographic limits of different production fluid formations that can be used by the system 400 or inhibitor model 430 to group together or collate the historical data 410. Using the associated performance data, the production fluids properties 414, and anti-agglomerant hydrate inhibitors properties 412 may be similar enough to allow the model to make high confidence level recommendations of anti-agglomerant hydrate inhibitors that will work effectively with the production fluid produced from that production fluid formation because of a larger available data set.

Other modules 444 may also include a communication module (not shown) allowing the inhibitor model 430 to communicate with historical data 410 that may be stored in the cloud, remote servers, or other remote computing devices. A communication module may also be used to allow remote access by users of the inhibitor model 430. Additional modules may include a search module that searches for data that may be used to update the historical data 410.

Input data 450 may be provided to the inhibitor model 430 including select target production fluid properties 452, inhibitor properties 454, use conditions 455, and user conditions 456. A user may want specific information about an anti-agglomerant hydrate inhibitor or type of anti-agglomerant hydrate inhibitor from the inhibitor model 430 and so may input select inhibitor properties 454. The information that a user may provide for the select inhibitor properties 454 may include the name of a specific anti-agglomerant hydrate inhibitor, its composition, and so forth. Further, the select inhibitor properties 454 may limit output to anti-agglomerant hydrate inhibitors above or below a threshold related but not limited to an anti-agglomerant hydrate inhibition efficiency, price, dosage, or availability.

The target production fluid properties 452 may include the name or other identifier of the target production fluid, a description of the source location, such as a geographic description, address, global positioning system coordinates, sections in a government survey system, the name or description of the geological formation that the target production fluid may be sourced from, and other geographically related properties of the target production fluid properties 452.

Use conditions 455 may also be described by a user. Use conditions 455 include but are not limited to environmental and field operating conditions. For example, use conditions 455 may include production fluid bulk temperature, pipeline outer wall temperature, weather conditions, flow rates, and pipeline and equipment materials and associated surface friction.

The user conditions 456 may specify the output data 460 that the user wants from the model. For example, the user may only want data, extrapolations, and predictions that have an effectiveness ranking above a certain threshold. The user may also specify how the recommendations should be ordered; alphabetically, by confidence level, by anti-agglomerant hydrate inhibition efficiency, by similarity to the target production fluid, etc.

The inhibitor model 430 may use an artificial intelligence model or a machine learning model to correlate the input data 450, such as the target production fluid properties 452 with production fluid properties 414 of the historical data 410. In one embodiment, the inhibitor model 430 may select a numerical tolerance to identify production fluids in the historical data 410 that may be similar to the target production fluid properties. For example, the inhibitor model 430 may provide a 1 percent tolerance for weight percent for anti-agglomerant hydrates in a production fluid in seeking to identify similar production fluids in the historical data 410. Further, the inhibitor model 430 may select a second or more properties to apply a numerical tolerance to for the identification of similar production fluids. These parameters may facilitate the identification of data that may be used to identify anti-agglomerant hydrate inhibitors having a high predicted anti-agglomerant hydrate inhibitor efficiency with the target production fluid.

The inhibitor model 430 may then use the correlated production fluid properties 414 to identify performance data 416, anti-agglomerant hydrate inhibitor properties 412, field information 418, and other information 419 within the historical data 410. The inhibitor model 430 may organize and output similar production fluid properties 462, recommended anti-agglomerant hydrate inhibitors 464 and their properties 466, the associated performance data 468, and other information 470 that may have been deemed relevant by the inhibitor model 430 or requested by a user. As previously discussed above, the inhibitor model 430 may extrapolate historical data 410 to identify unknown parameters in a data set of the historical data 410 to output data including one or more of a list of production fluids having one or more properties within a numerical tolerance of the properties of the target production fluid, a list of anti-agglomerant hydrate inhibitors for use with the target production fluid; and a list of inhibitor properties whose inhibitor effectiveness is above a threshold.

The output data 460 may potentially include a custom anti-agglomerant hydrate inhibitor not based on a currently available inhibitor. For example, the inhibitor model 430 may recommend mixing 4 or more known anti-agglomerant hydrate inhibitors at a specific ratio or percentage in order to achieve a superior anti-agglomerant hydrate inhibitor efficiency. Alternatively, the output data 460 may include a set of properties for a custom anti-agglomerant hydrate inhibitor based on the correlations identified by the inhibitor model 430.

The output data 460 may provide the inhibitor properties whose anti-agglomerant hydrate inhibitor effectiveness is above a numerical threshold, which may be provided by the user or predetermined. The model may also use parameter importance established during model building, where the parameter importance indicates dependence of inhibitor effectiveness rating, and the model may define a parameter importance score based on the parameter importance. Parameters may refer to the properties of the anti-agglomerant hydrate inhibitors, production fluids, and performance data that the inhibitor model 430 correlates with a predicted high anti-agglomerant hydrate inhibitor efficiency for a particular production fluid.

Further, the inhibitor model 430 may perform response surface modeling or other sensitivity analysis to determine variables of inhibitor structure to which inhibition effectiveness is most effective for a target production fluid. Additionally, or alternately, parameter importance to the effectiveness rating can be identified during model building and used in design or procurement of anti-agglomerant hydrate inhibitors.

The inhibitor model 430 may also base its recommendations on parameters such as a product catalog 422 or other market data available in the field information 418. For example, the inhibitor model 430 may output a recommendation that notes the cost of precursors, or manufacturing methods and or equipment for a particular anti-agglomerant hydrate inhibitor may be unusually expensive or difficult to obtain or use so that implementation would be cost prohibitive. The target production fluid properties 452 may be input to include a cost threshold that may instruct the inhibitor model 430 to remove potential anti-agglomerant hydrate inhibitors from the output if the cost to produce or the precursors are sold at a price high enough to make the use of that anti-agglomerant hydrate inhibitor cost prohibitive in spite of having a high anti-agglomerant hydrate inhibiting effectiveness and confidence level. Similarly, the dosage of an anti-agglomerant hydrate inhibitor may be used to increase or decrease the overall effectiveness rating or placement of the anti-agglomerant hydrate inhibitor in the listing of recommendations if the dosage is too high to be economically feasible.

The inhibitor model 430 may limit recommendations to off-the-shelf anti-agglomerant hydrate inhibitors that are currently stocked in sufficient quantities by commercial entities that regularly upload their inventory data to the historical data 410. In other cases, the inhibitor model 430 may output production fluids similar to the target production fluid, based on a numerical score computed from numerically described properties of the input production fluid, along with all inhibitors tested with that production fluid in the historical data 410, and their test results. The inhibitor model 430 may be configured to output an anti-agglomerant hydrate inhibitor functionality type predicted to be most effective for use with the target production fluid.

Once the inhibitor model 430 has output data 460, a user may test one or more of the anti-agglomerant hydrate inhibitor recommendations with the target production fluid 480 to determine its actual anti-agglomerant hydrate inhibition efficiency or effectiveness. Alternatively, the inhibitor model 430 may also specify testing parameters as part of the output data to validate the model’s extrapolations and predictions. The resulting test data may be used to make a final selection of the anti-agglomerant hydrate inhibitor and associated dosage 482. Further, the resulting test data may be input into the historical data 410. The new data may be used to recalibrate and re-train the inhibitor model 430. The historical data 410 may also be used to update the inhibitor model 430 on a regular periodic basis or only when a certain threshold of new data or certain types of new data are added to the historical data 410. For example, the inhibitor model 430 may be recalibrated and re-trained when test data for previously recommended anti-agglomerant hydrate inhibitors for a target production fluid 480 are input into the historical data 410 or when the final selection of an anti-agglomerant hydrate inhibitor and its associated dosage for a target production fluid is made 482 and this information is input into the historical data 410. Alternatively, the inhibitor model 430 may be recalibrated and re-trained when a report of observations of the actual usage of the anti-agglomerant hydrate inhibitor at the recommended dosage in a target production fluid within equipment is input into the historical data.

To calibrate the inhibitor model 430, a calibration module 432 may tune the relationships between parameters of the historical data 410 and the inhibitor model 430 until the output data are consistent with test results from recommended anti-agglomerant hydrate inhibitor tests with a target production fluid. In some embodiments, the parameters of the historical data 410 and the inhibitor model 430 may be calibrated to minimize the differences between the extrapolated and predicted values versus the actual test results.

The inhibitor model 430 further includes a weight module 433. The weight module 433 may assign a weight to one or more ranking factors used by the ranking module 438. For example, the weight module 433 may assign a weight to the water cut of the production fluid, the API gravity of the production fluid, the salinity of the production fluid, number of tests performed on the production fluid, the longest distance of the fluid properties from the historical fluid properties (e.g., the distance or difference between the measured fluid properties of the production fluid sample and the fluid properties of a tested sample in the historical data 410), any other weight, and combinations thereof.

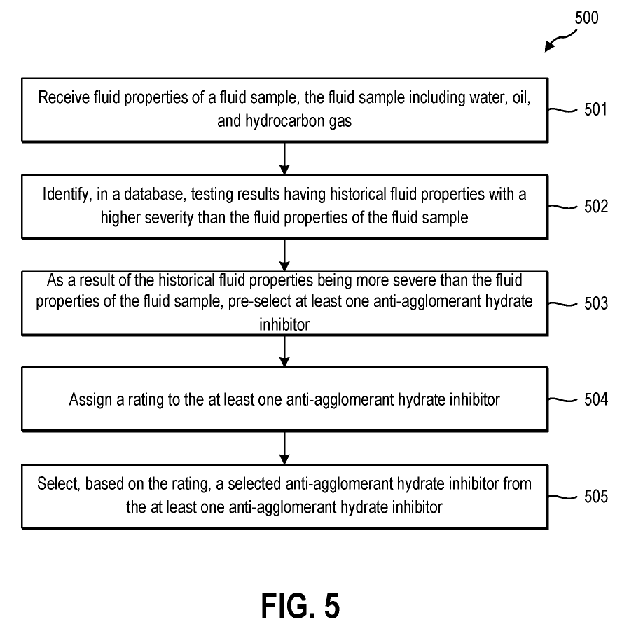

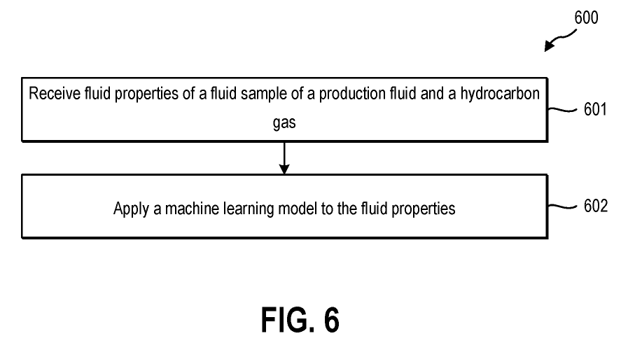

FIGS. 5 and FIG. 6, the corresponding text, and the examples provide a number of different methods, systems, devices, and computer-readable media of the anti-agglomerant hydrate inhibitor selection system. In addition to the foregoing, one or more embodiments can also be described in terms of flowcharts comprising acts for accomplishing a particular result, as shown in FIGS. 5 and FIG. 6. FIGS. 5 and FIG. 6 may be performed with more or fewer acts. Further, the acts may be performed in differing orders. Additionally, the acts described herein may be repeated or performed in parallel with one another or parallel with different instances of the same or similar acts.

As mentioned, FIG. 5 illustrates a flowchart of a series of acts or a method 500 for selecting anti-agglomerant hydrate inhibitors for a target production fluid, according to at least one embodiment of the present disclosure. While FIG. 5 illustrates acts according to one embodiment, alternative embodiments may omit, add to, reorder, and/or modify any of the acts shown in FIG. 5. The acts of FIG. 5 can be performed as part of a method. Alternatively, a computer-readable medium can comprise instructions that, when executed by one or more processors, cause a computing device to perform the acts of FIG. 5. In some embodiments, a system can perform the acts of FIG. 5.

An inhibitor model may receive fluid properties of a fluid sample at 501. The fluid sample includes water, oil, and hydrocarbon gas at 501. The inhibitor model may identify, in a database, testing results having historical fluid properties with a higher severity than the fluid properties of the fluid sample at 502. The testing results include tests of the historical fluid properties tested with a plurality of anti-agglomerant hydrate inhibitors. The inhibitor model may, as a result of the historical fluid properties being more severe than the fluid properties of the fluid sample, the inhibitor model may pre-select at least one anti-agglomerant hydrate inhibitor at 503. For example, as discussed herein, the historical fluid properties may be more severe than the fluid properties of the fluid sample when the historical fluid properties are more prone to develop hydrate agglomerates or develop larger hydrate agglomerates. The inhibitor model may assign a rating to the at least one anti-agglomerant hydrate inhibitor at 504. The inhibitor model may select, based on the rating, a selected anti-agglomerant hydrate inhibitor from the at least one anti-agglomerant hydrate inhibitor.

As mentioned, FIG. 6 illustrates a flowchart of a series of acts or a method 600 for selecting anti-agglomerant hydrate inhibitors for a target production fluid, according to at least one embodiment of the present disclosure. While FIG. 6 illustrates acts according to one embodiment, alternative embodiments may omit, add to, reorder, and/or modify any of the acts shown in FIG. 6. The acts of FIG. 6 can be performed as part of a method. Alternatively, a computer-readable medium can comprise instructions that, when executed by one or more processors, cause a computing device to perform the acts of FIG. 6. In some embodiments, a system can perform the acts of FIG. 6.

An anti-agglomerant hydrate inhibitor selection system receives fluid properties of a fluid sample of a production fluid including water, oil, and a hydrocarbon gas at 601. A machine learning model is applied to the fluid properties at 602. The machine learning model is trained to generate an output of at least one anti-agglomerant hydrate inhibitor based on an input of fluid properties. The machine learning model outputs a list of anti-agglomerant hydrate inhibitors for the fluid sample based on the fluid properties.

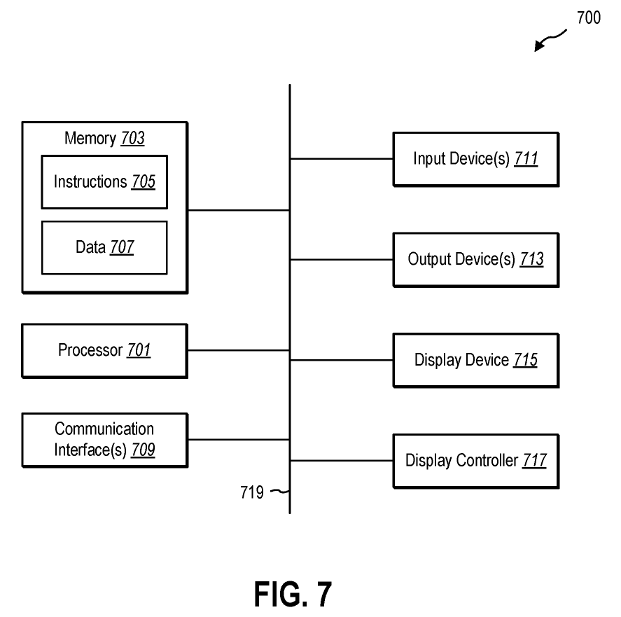

FIG. 7 illustrates certain components that may be included within a computer system 700. One or more computer systems 700 may be used to implement the various devices, components, and systems described herein.

The computer system 700 includes a processor 701. The processor 701 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 701 may be referred to as a central processing unit (CPU). Although just a single processor 701 is shown in the computer system 700 of FIG. 7, in an alternative configuration, a combination of processors (e.g., an ARM and DSP) could be used.

The computer system 700 also includes memory 703 in electronic communication with the processor 701. The memory 703 may be any electronic component capable of storing electronic information. For example, the memory 703 may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 705 and data 707 may be stored in the memory 703. The instructions 705 may be executable by the processor 701 to implement some or all of the functionality disclosed herein. Executing the instructions 705 may involve the use of the data 707 that is stored in the memory 703. Any of the various examples of modules and components described herein may be implemented, partially or wholly, as instructions 705 stored in memory 703 and executed by the processor 701. Any of the various examples of data described herein may be among the data 707 that is stored in memory 703 and used during execution of the instructions 705 by the processor 701.

A computer system 700 may also include one or more communication interfaces 709 for communicating with other electronic devices. The communication interface(s) 709 may be based on wired communication technology, wireless communication technology, or both. Some examples of communication interfaces 709 include a Universal Serial Bus (USB), an Ethernet adapter, a wireless adapter that operates in accordance with an Institute of Electrical and Electronics Engineers (IEEE) 802.11 wireless communication protocol, a Bluetooth^{®} wireless communication adapter, and an infrared (IR) communication port.

A computer system 700 may also include one or more input devices 711 and one or more output devices 713. Some examples of input devices 711 include a keyboard, mouse, microphone, remote control device, button, joystick, trackball, touchpad, and lightpen. Some examples of output devices 713 include a speaker and a printer. One specific type of output device that is typically included in a computer system 700 is a display device 715. Display devices 715 used with embodiments disclosed herein may utilize any suitable image projection technology, such as liquid crystal display (LCD), light-emitting diode (LED), gas plasma, electroluminescence, or the like. A display controller 717 may also be provided, for converting data 707 stored in the memory 703 into text, graphics, and/or moving images (as appropriate) shown on the display device 715.

The various components of the computer system 700 may be coupled together by one or more buses, which may include a power bus, a control signal bus, a status signal bus, a data bus, etc. For the sake of clarity, the various buses are illustrated in FIG. 7 as a bus system 719.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers’ specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that is within standard manufacturing or process tolerances, or which still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for selecting an anti-agglomerant hydrate inhibitor, the method comprising:
receiving fluid properties of a fluid sample, the fluid sample including water and hydrocarbons;
identifying, testing results having historical fluid properties with a higher severity than the fluid properties of the fluid sample, the testing results including tests of the historical fluid properties tested with a plurality of anti-agglomerant hydrate inhibitors;
as a result of the historical fluid properties being more severe than the fluid properties of the fluid sample, pre-selecting at least one anti-agglomerant hydrate inhibitor;
assigning a rating to the at least one anti-agglomerant hydrate inhibitor; and
selecting, based on the rating, a selected anti-agglomerant hydrate inhibitor from the at least one anti-agglomerant hydrate inhibitor.

2. The method of claim 1, wherein the fluid properties include water cut, API gravity, and salinity.

3. The method of claim 1, wherein the selected anti-agglomerant hydrate inhibitor includes a plurality of selected anti-agglomerant hydrate inhibitors, and wherein assigning the rating includes rating the plurality of selected anti-agglomerant hydrate inhibitors based on a dosage of a tested anti-agglomerant hydrate inhibitor, a number of tests of the tested anti-agglomerant hydrate inhibitor, and a distance of the fluid properties of the fluid sample from the historical fluid properties.

4. The method of claim 3, further comprising assigning a weight to each of the dosage of the tested anti-agglomerant hydrate inhibitor, the number of tests of the tested anti-agglomerant hydrate inhibitor, and the distance of the fluid properties of the fluid sample from the historical fluid properties.

5. The method of claim 3, further comprising selecting a chosen anti-agglomerant hydrate inhibitor from the plurality of selected anti-agglomerant hydrate inhibitors based on the rating.

6. The method of claim 1, wherein, when the rating is above a threshold, selecting the selected anti-agglomerant hydrate inhibitor includes selecting the selected anti-agglomerant without testing the tested anti-agglomerant hydrate inhibitor.

7. The method of claim 1, further comprising training a machine learning model on the testing results, the machine learning model trained to pre-select the at least one anti-agglomerant hydrate inhibitor.

8. The method of claim 7, wherein training the machine learning model includes training the machine learning model to assign the rating to the at least one anti-agglomerant hydrate inhibitor.

9. The method of claim 7, wherein training the machine learning model includes training the machine learning model to select the selected anti-agglomerant hydrate inhibitor.

10. The method of claim 7, wherein training the machine learning model includes training the machine learning model to assign the rating based on a dosage of a tested anti-agglomerant hydrate inhibitor, a number of tests of the tested anti-agglomerant hydrate inhibitor, and a distance of the fluid properties of the fluid sample from the historical fluid properties.

11. The method of claim 10, further comprising fine-tuning the machine learning model by adjusting a weight of a dosage of the tested anti-agglomerant hydrate inhibitor, the number of tests of the tested anti-agglomerant hydrate inhibitor, and the distance of the fluid properties of the fluid sample from the historical fluid properties.

12. A method, comprising:
receiving fluid properties of a fluid sample of a production fluid, oil, and a hydrocarbon gas;
applying a machine learning model to the fluid properties, wherein the machine learning model is configured to generate an output of at least one anti-agglomerant hydrate inhibitor based on an input of fluid properties, wherein the machine learning model outputs a list of anti-agglomerant hydrate inhibitors for the fluid sample based on the fluid properties; and
training the machine learning model based on at least one of the list of anti-agglomerant hydrate inhibitors.

13. The method of claim 12, further comprising selecting or designing a chosen anti-agglomerant hydrate inhibitor, based on the output, for use with the fluid sample.

14. The method of claim 13, further comprising testing the chosen anti-agglomerant hydrate inhibitor with the fluid sample.

15. The method of claim 14, wherein training the machine learning model includes adjusting a weight of one or more testing parameters based on the testing of the chosen anti-agglomerant hydrate inhibitor.

16. The method of claim 15, wherein training the machine learning model includes training the machine learning model to assign a rating based on a dosage of the chosen anti-agglomerant hydrate inhibitor, a number of tests of the chosen anti-agglomerant hydrate inhibitor, and a distance of the fluid properties of the fluid sample from historical fluid properties.

17. The method of claim 12, further comprising generating an effectiveness rating for each of the list of anti-agglomerant hydrate inhibitors, the list of anti-agglomerant hydrate inhibitors ranked by the effectiveness rating.

18. The method of claim 12, wherein the machine learning model outputs an effectiveness rating for the at least one anti-agglomerant hydrate inhibitors of the list of anti-agglomerant hydrate inhibitors.

19. The method of claim 12, wherein the machine learning model is trained on historical data including a plurality of anti-agglomerant hydrate inhibitor tests, the plurality of anti-agglomerant hydrate inhibitor tests testing a tested anti-agglomerant hydrate inhibitor on a sample.

20. A system, comprising
a processor; and
memory, the memory including instructions that cause the processor to:
receive fluid properties of a fluid sample, the fluid sample including water, oil, and hydrocarbon gas;
identify testing results having historical fluid properties with a higher severity than the fluid properties of the fluid sample, the testing results including tests of the historical fluid properties tested with a plurality of anti-agglomerant hydrate inhibitors;
as a result of the historical fluid properties being more severe than the fluid properties of the fluid sample, pre-select at least one anti-agglomerant hydrate inhibitor;
assign a rating to the at least one anti-agglomerant hydrate inhibitor; and
select, based on the rating, a selected anti-agglomerant hydrate inhibitor from the at least one anti-agglomerant hydrate inhibitor.
